(19) United States
(12) Patent Application Publication
(10) Pub. No.: US 2020/0093466 A1
(43) Pub. Date: Mar. 26, 2020

(54) URINE SPECIMEN CUP HOLDER

(71) Applicant: Charles M. Washington, Roscoe, IL (US)

(72) Inventor: Charles M. Washington, Roscoe, IL (US)

(21) Appl. No.: 16/559,925

(22) Filed: Sep. 4, 2019

Related U.S. Application Data
(63) Continuation of application No. 29/664,576, filed on Sep. 26, 2018.
(60) Provisional application No. 62/736,860, filed on Sep. 26, 2018.

(51) Int. Cl. A61B 10/00; B01L 9/00

(52) U.S. Cl. CPC A61B 10/007; B01L 9/00

(57) ABSTRACT

A urine specimen cup holder is provided to collect a urine stream in a specimen collection cup. The urine specimen cup holder includes a handle and a receiver portion at an end of the handle. The receiver portion is sized to at least partially surround and removably hold the urine specimen cup.

### CROSS-REFERENCE TO RELATED PATENT APPLICATIONS

This patent application claims the benefit of U.S. Provisional Patent Application No. 62/736,860, filed Sep. 26, 2018, and U.S. Design patent application Ser. No. 29/664,576, filed Sep. 26, 2018, the entire teachings and disclosures of which are incorporated herein by reference thereto.

### FIELD OF THE INVENTION

This invention generally relates to the medical device field, more particularly to urine collection devices used in a diagnostic urinalysis testing.

### BACKGROUND OF THE INVENTION

Medical providers often order a patient to submit to a urinalysis test in order to help the provider reach a diagnosis of the patient's condition and/or to assess the patient's overall health. The patient is asked to obtain a “mid-stream” urine sample at an intermittent stage of the urination event. Such “mid-stream” collection avoids, to the extent possible, extraneous debris in the sample.

However, traditional means of obtaining the urine for the urinalysis testing can be problematic for certain patients. For example, it is fairly universal for medical providers to provide a urine specimen cup, also referred to as a urine collection cup. The urine specimen cup typically has a standard 120 cubic centimeter (cc)/4 ounce (oz.) volume. Typical dimensions of the urine specimen cup are 56.8 mm in outer diameter by 72.5 mm in height with a cap outer dimension of 61 mm. Such urine specimen cups are shown in FIG. 4 labeled prior art.

Other efforts to address urine collection have also been tried, for example in the urine stream collection unit disclosed in U.S. patent application number 2009/0118642 to Charles M. Washington, inventor of the instant Application. However, the device disclosed therein, while functional, has the disadvantage of not being able to accommodate the standard 120 cc urine specimen cup/collection bottle that is in widespread use at medical facilities and by a majority of medical providers. Further, one using the device disclosed therein has to pour the urine sample into the 120 cc urine specimen cup or like sample/specimen bottles for delivery to and testing by a lab. This transfer after collection presents an undesired possibility for contamination.

Accordingly, some patients are still presented with difficulties in attempting to hygienically collect their urine stream using the 120 cc urine specimen cups or similar collection bottles. Arthritic patients, patients having difficulty with their limbs, the very obese, pregnant patients and those with various other disabilities may have a very difficult time reaching the area between their legs near the urethra so as to properly place the urine specimen cup to collect the urine sample.

### BRIEF SUMMARY OF THE INVENTION

Embodiments of the invention provide a urine specimen cup holder having a handle and a receiver portion at the end of the handle that can receive and hold a standard 120 cc urine specimen cup for collecting urine specimens. Using this elegant and innovative device provides a simple means by which patients with disabilities, obesity, pregnancy or other disabling conditions can position the urine specimen cup between their legs proximate their urethra to collect a urine specimen. Accordingly, the use of the urine specimen cup holder avoids soiling of the hands and inner thighs with urine because it permits the collection bottle to be placed proximate the urethra to collect the stream instead of having patients use only the urine specimen cup/bottle without the handle which causes them to strain and to guess at a position of collection which can result in a contaminated specimen which is not useful for medical testing.

Embodiments of the urine specimen cup holder include a handle which can have various lengths or can even be extendable to provide the desired length to reach the patient's urethra, depending on their needs. Once the patient is given the urine specimen cup he or she can simply drop/insert the urine specimen cup in the receiver portion of the urine specimen cup holder. In certain embodiments, the receiver portion has a first and second arms that are along a circular outer circumference. The arms are separated by a gap. Each of the arms of the receiver portion have an inner cylindrical surface that may be smooth and that that bears against the urine specimen cup's outer cylindrical surface below its threads to provide a snug fit for the urine specimen cup to minimize movement of the urine specimen cup while at the same time allowing the urine specimen cup to be easily inserted and removed therefrom. In certain embodiments, the receiver portion partially surrounds the urine specimen cup when the urine specimen cup is inserted into the aperture defined by the receiver portion, more specifically defined by its arms. The cylindrical receiver portion, in an embodiment can be an endless circle in cross section to removably hold and completely surround a standard size 120 cc urine specimen cup. By removably hold it is meant that the urine specimen cup is secure enough within the cylindrical receiver portion to be moved by the user to collect the mid-stream urine sample and then easily removed from the cylindrical receiver portion after the sample has been taken.

Further, embodiments provide that the cylindrical receiver portion can have a gap such that urine specimen cups having a circumference slightly larger than that of a standard 120 cc urine specimen cup can be accommodated by the cylindrical receiver portion because the gap allows for the diameter of the cylindrical receiver portion to vary because of the gap and the elasticity provided by the materials of its construction, for example a thermoplastic construction. Such a gap can also reduce production costs because of the reduced material requirements.

Embodiments of the invention also provide for a method of collection of urine for urinalysis. The method includes the steps of directing a patient to insert a urine specimen cup into the cylindrical receiver portion of a handle. The directions can be verbal as for example a doctor to a patient or written as in schematic drawings or explicit directions. The method further includes the step of directing the patient to position the cylindrical receiver portion proximate the urethra to collect a urine stream specimen. By proximate it is meant generally in the area between the knees and the urethra, preferably closer to the urethra than to the knees. The method further includes collecting a urine stream specimen into the urine specimen cup and then sealing, that is, closing the urine specimen cup with a cap.

In one aspect, an embodiment of the invention provides a urine specimen cup holder to collect a urine stream in a urine specimen cup. The urine specimen cup holder includes a handle and a receiver portion at an end of the handle. The receiver portion is sized to at least partially surround and removably hold the urine specimen cup.

In an embodiment, the receiver portion has an inner most diameter between 55 and 59 mm.

In an embodiment, the receiver portion has a first circumferential end and a second circumferential end and a gap between the first circumferential end and the second circumferential end.

In an embodiment, a circumferential distance of the gap is less than half of a total circumferential distance around the receiver portion.

In an embodiment, the receiver portion is an endless circle.

In an embodiment, an innermost circumferential surface of the receiver portion is smooth and is configured to contact an outer peripheral surface of the urine specimen cup that is smooth.

In an embodiment, an innermost diameter of the receiver portion is less than an outermost diameter of the urine specimen cup.

In another aspect, an embodiment of the invention provides a urine specimen cup holder assembly. The urine specimen cup assembly includes a urine specimen cup holder which includes a handle and a receiver portion at an end of the handle. The urine specimen cup holder assembly also includes a urine specimen cup. The receiver portion is sized to at least partially surround and hold the urine specimen cup.

In an embodiment, the urine specimen cup is a 120 cc urine specimen cup.

In an embodiment, the receiver portion removably holds the urine specimen cup.

In an embodiment, the receiver portion is fixedly attached to the urine specimen cup.

In an embodiment, an outer diameter of the urine specimen cup in contact with the receiver portion is approximately 57 mm and the height is approximately 73 mm.

In an embodiment, the end of the handle is a second end and wherein a first end of the handle is sized to be held in a human hand.

In an embodiment, the handle is extendable to increase the length of the handle between the first end and the second end.

In an embodiment, the urine specimen cup holder is made of plastic.

In yet another aspect, an embodiment of the invention provides a method for collection of a mid-stream urine sample for urinalysis testing. The method includes the step of directing a user to place a urine specimen cup in urine specimen cup holder having a handle and a receiver portion at an end of the handle. The method includes the step of directing the user to collect the user's urine into the urine specimen cup removably held by the urine specimen cup holder.

In an embodiment, the method includes the step of inserting a bottom of the urine specimen cup through an aperture defined by the receiver portion.

In an embodiment, the method includes the step of collecting the mid-stream urine sample while the urine specimen cup is removably held by the urine specimen cup holder.

In an embodiment, the method includes the step of securing a cap to the urine specimen cup to close the urine specimen cup.

In an embodiment, the method includes the step of removing the closed urine specimen cup from the receiver portion.

Other aspects, objectives and advantages of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention. In the drawings:

FIG. 1 is a perspective view of a urine specimen cup holder according to the teachings of embodiments of the instant application; and

FIG. 2 is a perspective view of the urine specimen cup holder holding a urine specimen cup.

FIG. 3 is another perspective view of the urine specimen cup holder holding a urine specimen cup with its cover removed.

FIG. 4 is a perspective view of a typical 120 cc urine specimen cup.

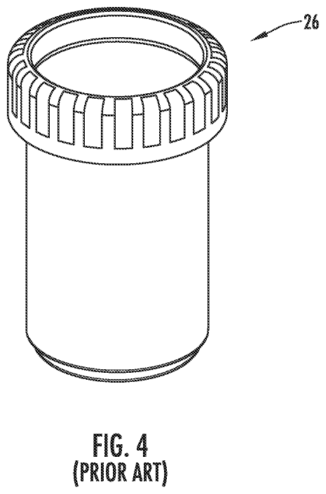

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

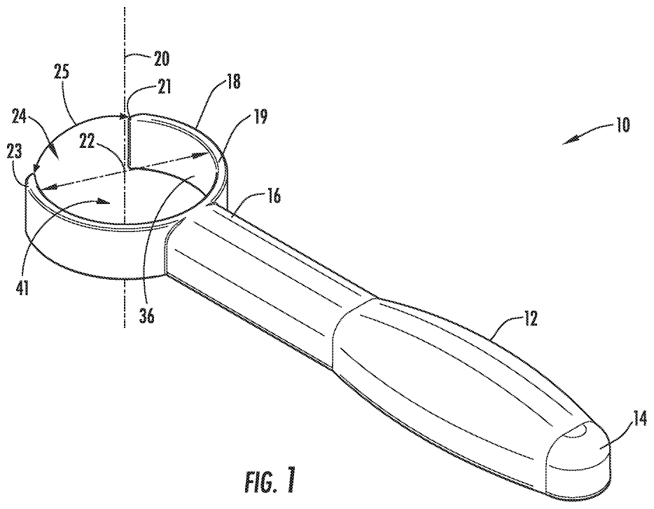

Turning now to the drawings, there is illustrated in FIG. 1 an exemplary embodiment of a urine specimen cup holder 10. While such an exemplary environment will be utilized in describing various features and advantages of embodiments of the invention, such a description should be taken by way of example and not by limitation. Indeed, advantages of embodiments of the invention can be used by patients to improve the holding and placement of urine specimen collection cups for medical testing.

In an embodiment a urine specimen cup holder 10 includes a handle 12 having a first end 14 and a second end 16. The second end 16 includes a receiver portion 18 that is generally cylindrical and extends away from first end 14 and the second end 16. The receiver portion 18 defines an aperture 41 having a central axis 20 and an inner most diameter 22 extending transversely through the central axis 20. Further, the receiver portion 18 has a gap 24 along its outer circumference 19. The gap 24 is between a first circumferential end 21 and a second circumferential end 23. The circumferential distance 25 of the gap 24 is less than half the circumferential distance around the receiver portion 18. The first end 14 is sized such that a human hand can easily hold the handle 12 proximate the first end 14.

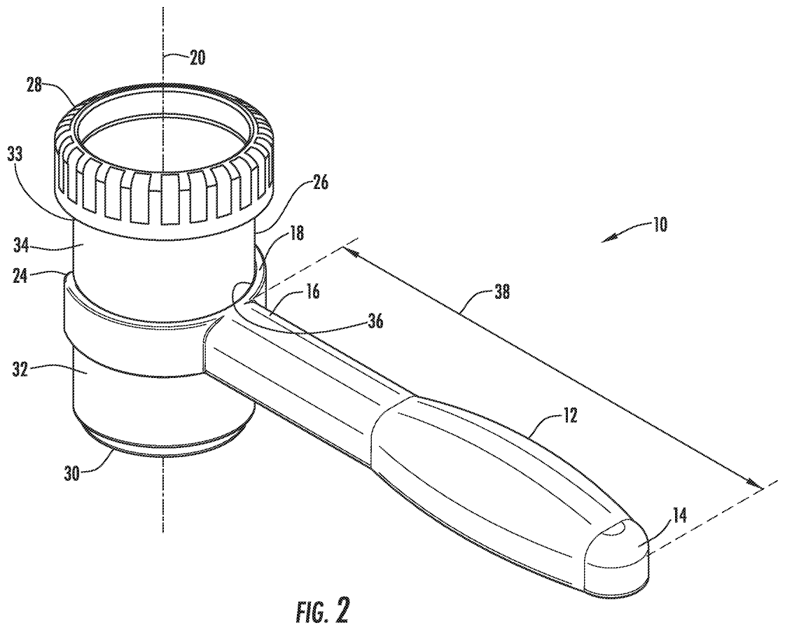

In an embodiment, the inner most peripheral surface 36 of the receiver portion 18 may be smooth. In yet other embodiments the inner most peripheral surface may include a friction surface, for example rubber to increase the friction between the inner most peripheral surface 36 and the outer peripheral surface 34 (FIG. 2) of the urine specimen cup 26.

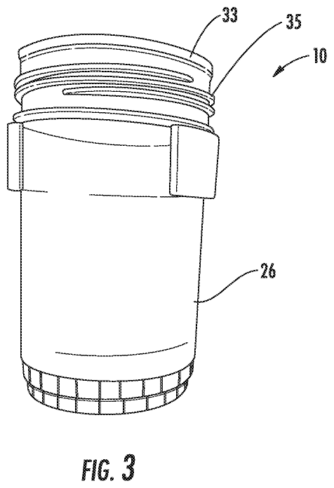

FIGS. 2 and 3 illustrate the urine specimen cup holder 10 with a urine specimen cup 26 with a cap 28 that closes an open top 33 of the urine specimen cup 26. The other end of the urine specimen cup 26 is a closed bottom 30 at the other end and cylindrical body portion 32 therebetween. In an embodiment the receiver portion 18 is sized to receive the urine specimen cup 26 that is a standard size 120 cc by inserting the closed bottom 30 of the urine specimen cup 26 in a direction of insertion parallel to the central axis 20 through the aperture 41.

By standard size it is meant the urine specimen cup 26 has a width/diameter of approximately 57 mm (millimeters) and a height of approximately 73 mm; the outermost diameter may be greater than 57 mm at the open top 33 of the urine specimen cup 26 where threads 35 are located for securing the cap 28. The cap 28 may have an outer diameter of approximately 61 mm. By sized to receive, it is mean that an outer peripheral surface 34 of the cylindrical body portion 32 fits snuggly against the inner most peripheral surface 36 of the receiver portion 18 such that the urine specimen cup 26 is held by the urine specimen cup holder 10 for positioning proximate the urethra of a user for the collection of a urine stream. Thus, the receiver portion 18 removably surrounds the cylindrical body portion 32 of the urine specimen cup 26. By removably surrounds it is meant that, although the fit is snug, the urine specimen cup 26 may be inserted and removed. They are not permanently fixed, monolithic or connected to one another. Because outermost diameter at the threads 35 of the open top 33 of the urine specimen cup 26 is greater than the inner most diameter 22 of the receiver portion 18 then the open top 33 of the urine specimen cup 26 cannot fall through the aperture 41 provided by the receiver portion 18.

In an embodiment, the receiver portion 18 may be comprised of a thermoplastic material that is flexible enough to increase the gap 24 sufficiently to accommodate the outer peripheral surface 34 of the urine specimen cup 26 that is standard size or may be sized to accommodate larger or smaller sizes with a fixed aperture 41 or with and adjustable aperture size so as to provide for a snug fit to hold the urine specimen cup 26. However, it can be readily appreciated that in yet other embodiments the receiver portion 18 does not have a gap 24 but is a continuous cylinder. Urine specimen cup holder 10, may be made of most any material available, including steels, plastics or composites by way of non-limiting examples.

In another embodiment the urine specimen cup holder 10 may be fixedly attached to the urine specimen cup 26.

While embodiments of the handle 12 may be of a length 38 that is fixed between the first end 14 and the second end 16, in yet other embodiments, the handle 12 may be extendable or retractable such that a patient or medical provider can increase or decrease the length 38 between the first end 14 and the second end 16 to facilitate the urine stream collection.

In use in and embodiment of the method, a patient or user who must provide a mid-stream urine sample for urinalysis testing is directed to place a urine specimen cup 26 in urine specimen cup holder 10 having a handle 12 and a receiver portion 18 at an end of the handle 12. The patient or provider or an agent of the provider such as a nurse, inserts the closed bottom 30 of the urine specimen cup 26 through the aperture 41 defined by the receiver portion 18.

The receiver portion is configured, as previously discussed such that the urine specimen cup will not fall out of the receiver portion 18 as the patient collects the mid-stream urine sample while the urine specimen cup. After the specimen is collected then then the user or provider secures the cap 28 to the urine specimen cup 26 on the open top 33 to close the urine specimen cup 26.

The cap 28 may be secured while the urine specimen cup 26 is in the receiver portion 18 or after removing the urine specimen cup 16 from the receiver portion 18 such that the closed urine specimen cup 26 may be sent to a medical lab for testing.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A urine specimen cup holder to collect a urine stream in a urine specimen cup, the urine specimen cup holder comprising:
a handle;
a receiver portion at an end of the handle;
wherein the receiver portion is sized to at least partially surround and removably hold the urine specimen cup.

2. The urine specimen cup holder of claim 1, wherein the receiver portion has an inner most diameter between 55 and 59 mm.

3. The urine specimen cup holder of claim 1, wherein the receiver portion has a first circumferential end and a second circumferential end and a gap between the first circumferential end and the second circumferential end.

4. The urine specimen cup holder of claim 3, wherein a circumferential distance of the gap is less than half of a total circumferential distance around the receiver portion.

5. The urine specimen cup holder of claim 1, wherein the receiver portion is an endless circle.

6. The urine specimen cup holder of claim 1, wherein an innermost circumferential surface of the receiver potion is smooth and is configured to contact an outer peripheral surface of the urine specimen cup that is smooth.

7. The urine specimen cup holder of claim 1, wherein an innermost diameter of the receiver portion is less than an outermost diameter of the urine specimen cup.

8. A urine specimen cup holder assembly, comprising:
a urine specimen cup holder comprising:
a handle;
a receiver portion at an end of the handle;
a urine specimen cup;
wherein the receiver portion is sized to at least partially surround and hold the urine specimen cup.

9. The urine specimen cup holder assembly of claim 8, wherein urine specimen cup is a 120 cc urine specimen cup.

10. The urine specimen cup holder assembly of claim 8, wherein the receiver portion removably holds the urine specimen cup.

11. The urine specimen cup holder assembly of claim 8, wherein the receiver portion is fixedly attached to the urine specimen cup.

12. The urine specimen cup holder assembly of claim 8, wherein a diameter of the urine specimen cup in contact with the receiver portion is approximately 57 mm and the height is approximately 73 mm.

13. The urine specimen cup holder assembly of claim 8, wherein the end of the handle is a second end and wherein a first end of the handle is sized to be held in a human hand.

14. The urine specimen cup holder assembly of claim 13, wherein the handle is extendable to increase the length of the handle between the first end and the second end.

15. The urine specimen cup holder assembly of claim 8, wherein the urine specimen cup holder is made of plastic.

16. A method for collection of a mid-stream urine sample for urinalysis testing, the method comprising the steps of:
directing a user to place a urine specimen cup in urine specimen cup holder having a handle and a receiver portion at an end of the handle; and
directing the user to collect the user's urine into the urine specimen cup removably held by the urine specimen cup holder.

17. The method of claim 16, further comprising the step of inserting a closed bottom of the urine specimen cup through an aperture defined by the receiver portion.

18. The method of claim 16, further comprising the step of collecting the mid-stream urine sample while the urine specimen cup is removably held by the urine specimen cup holder.

19. The method of claim 16, further comprising the step of securing a cap to an open top of the urine specimen cup to close the urine specimen cup.

20. The method of claim 16, further comprising the step of removing the closed urine specimen cup from the receiver portion.
